(19) United States
(12) Patent Application Publication
(10) Pub. No.: US 2010/0282262 A1
(43) Pub. Date: Nov. 11, 2010

(54) METHOD AND DEVICE FOR RESUSCITATING A PERSON IN CARDIAC ARREST CONDITION

(76) Inventor: Georges Boussignac, Antony (FR)

Correspondence Address: Dickinson Wright PLLC;James E. Ledbetter, Esq., International Square, 1875 Eye Street, N.W., Suite 1200, Washington, DC, 20006, US

(21) Appl. No.: 12/527,398

(22) PCT Filed: Feb. 8, 2008
(86) PCT No.: PCT/FR08/00153

§ 371 Date: Aug. 14, 2009

(30) Foreign Application Priority Data
Feb. 15, 2007 (FR) 07/01,087

(51) Int. Cl. A61M 16/20

(52) U.S. Cl. 128/205.24

(57) ABSTRACT

The invention relates to the use of a device that comprises a tubular member (5), auxiliary channels (9) continuously injecting jets of respiratory gases, deflexion means (8) for converging said respiratory gas jets inside the tubular member (5), and means (S1) for slowing down the inflow of outside air into the tubular member (5) at the beginning of each decompression imposed on the thoracic cage of a person.

The present invention relates to a method and a device for resuscitating persons in a state of cardiac arrest.

When trying to resuscitate a person who is in a state of cardiac arrest, it is known to exert alternate cycles of compression and decompression on the thoracic cage of this person in an attempt to restore the movements of exhalation and inhalation.

Moreover, for example, European patents EP 0 390 684 and EP 0 911 051 have already disclosed a device for respiratory assistance of patients whose breathing, although occurring spontaneously, is insufficient. A known device of this kind for respiratory assistance comprises:
a tubular element which forms a main channel and which is designed to be connected via its distal end to an airway of a patient, while the proximal end of said tubular element is located outside said patient, and the latter's respiratory system is connected to the outside by way of said main channel;
peripheral auxiliary channels which open into said main channel, said auxiliary channels being supplied continuously with respiratory gas; and
deflection means for causing the jets of respiratory gas injected through said auxiliary channels to converge on one another inside said main channel.

The patient is thus ventilated continuously by said jets of respiratory gas.

The Applicant has found that this device for respiratory assistance of a patient with spontaneous breathing can be successfully used as a device for artificial respiration (and not only as a device for respiratory assistance) on persons who are in a state of cardiac arrest and who are being resuscitated by alternate compressions and decompressions of their thoracic cage, the jets of said respiratory gas promoting the restoration of inhalation and circulation of the blood.

However, the Applicant has noted that said respiratory gas, introduced continuously into the lungs of the person in the state of cardiac arrest, generates a residual positive pressure in the lungs, at the end of a compression and at the beginning of the following decompression, and that this residual positive pressure continues during part of said decompression, before disappearing and being replaced by a negative pressure generated by the decompression. This residual positive pressure on the one hand forms an obstacle to the inspiration of outside air through said tubular element and on the other hand is maintained by said outside air that is inspired. This has the effect that, during a substantial part of each decompression, the lungs of said person do not efficiently inspire the outside air, and the circulation of blood (particularly the venous return) to the extremities (head, arms, legs) of said person is not satisfactory.

The object of the present invention is to overcome this disadvantage.

To this end, according to the invention, the method for resuscitating a person in a state of cardiac arrest, according to which method alternate compressions and decompressions are exerted on the thoracic cage of said person, is characterized in that:
a device is used that comprises:
   a tubular element which forms a main channel,
   peripheral auxiliary channels which open into said main channel, said auxiliary channels being supplied continuously with respiratory gas, and
   deflection means for causing the jets of respiratory gas injected through said auxiliary channels to converge on one another inside said main channel,
in such a way that said main channel is connected via its distal end to an airway of said person, while the proximal end of said tubular element is located outside said person, such that the latter's respiratory system is connected to the outside by way of said main channel, and
the intake of outside air into said tubular element is slowed down at the beginning of each decompression.

Thus, by virtue of the present invention, said residual positive pressure rapidly disappears under the action of the decompression, during the gradual intake of the outside air that is inspired. Said residual positive pressure therefore no longer constitutes an obstacle to the intake of outside air and to the circulation of blood of the person in cardiac arrest.

The device used according to the invention thus comprises means for slowing down the intake of air into said tubular element.

Advantageously, said means for slowing down the intake of outside air into said tubular element comprise a hollow body provided with first and second valves which are normally closed, said first valve being able to open spontaneously and immediately during the compressions, while said second valve is able to open spontaneously, but gradually, during the decompressions, and said hollow body being arranged at the proximal end of said tubular element.

Said first and second valves can be arranged either in parallel or in series between the outside and the internal cavity of said hollow body, that is to say between the outside and the interior of said tubular element (that is to say the lungs of the person being resuscitated).

In the case where said first and second valves are arranged in series, one of them can be supported by the other. It is then advantageous, on the one hand, that said first valve is formed by an elastic membrane bearing spontaneously against a seat provided in said hollow body and being connected to said seat via several attachment points distributed about its periphery, and the air expelled during the compressions passes freely from the inside of the hollow body to the outside via passages which form spontaneously and immediately through the elastic deformation of said membrane between said points of attachment and said seat, and, on the other hand, that said second valve is formed by at least one slit with contiguous edges made in said membrane, and the air inspired during the decompressions passes gradually from the outside to the interior of the hollow body via the passage which forms spontaneously in said membrane by elastic deformation thereof, causing its contiguous edges to move gradually apart.

Said means for slowing down the intake of outside air into said tubular element can form an integral part of the latter or can form a component that is able to be attached releasably to said tubular element.

The figures in the attached drawing will make it clear how the invention can be realized. In these figures, identical reference signs designate similar elements.

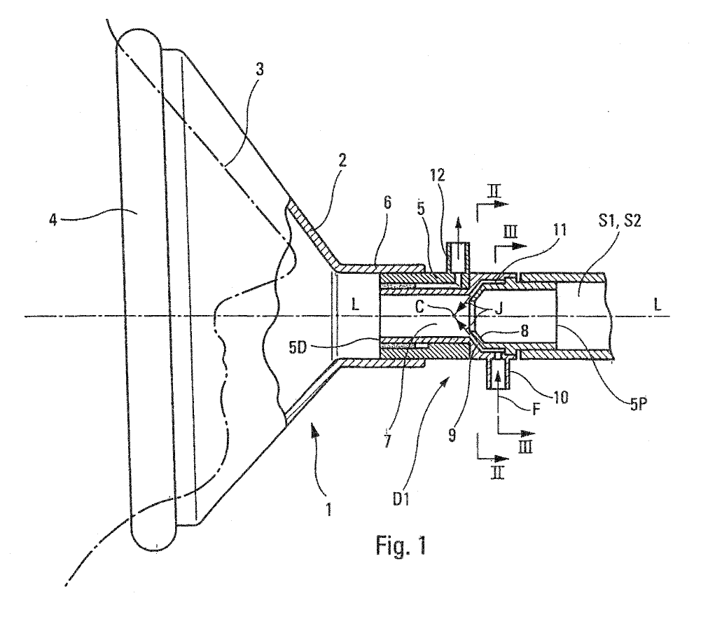

FIG. 1 is a schematic view, partially in axial section, of a breathing mask equipped with a first illustrative embodiment of the device for artificial respiration which is intended to be improved in accordance with the present invention.

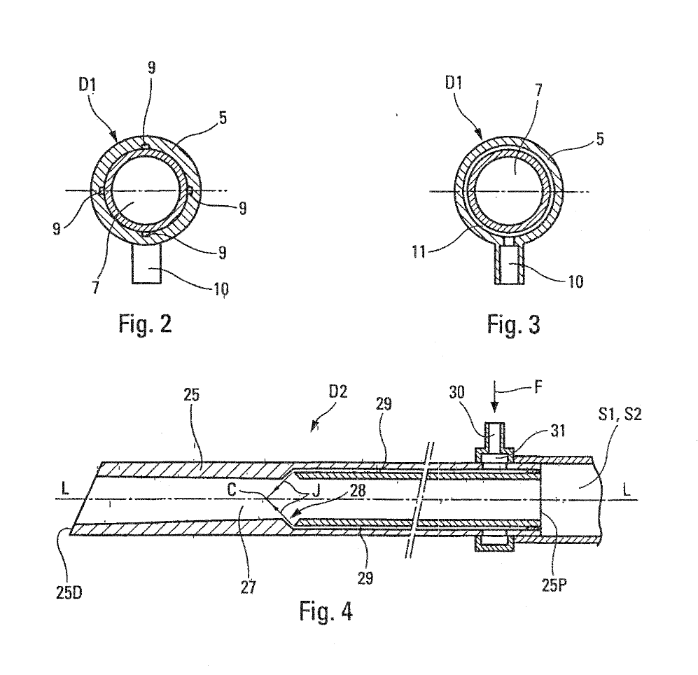

FIGS. 2 and 3 are transverse sections of the device for artificial respiration from FIG. 2 along the lines II-II and respectively.

FIG. 4 shows, in schematic longitudinal section, a second illustrative embodiment of the device for artificial respiration which is intended to be improved in accordance with the present invention.

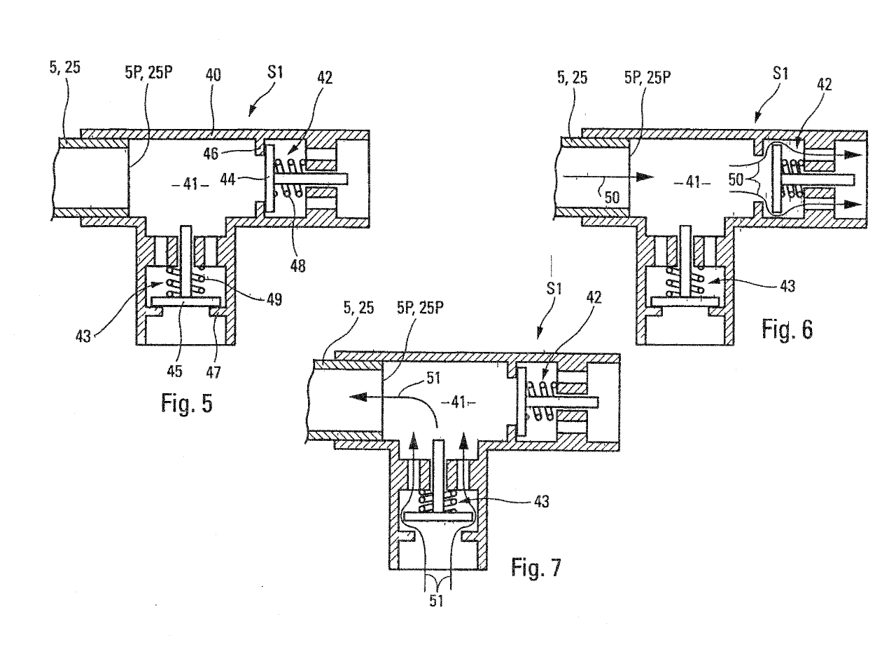

FIGS. 5, 6 and 7 illustrate, in schematic cross section, three states of a first example of a system according to the present invention, intended to improve the devices for artificial respiration from FIGS. 1 and 4.

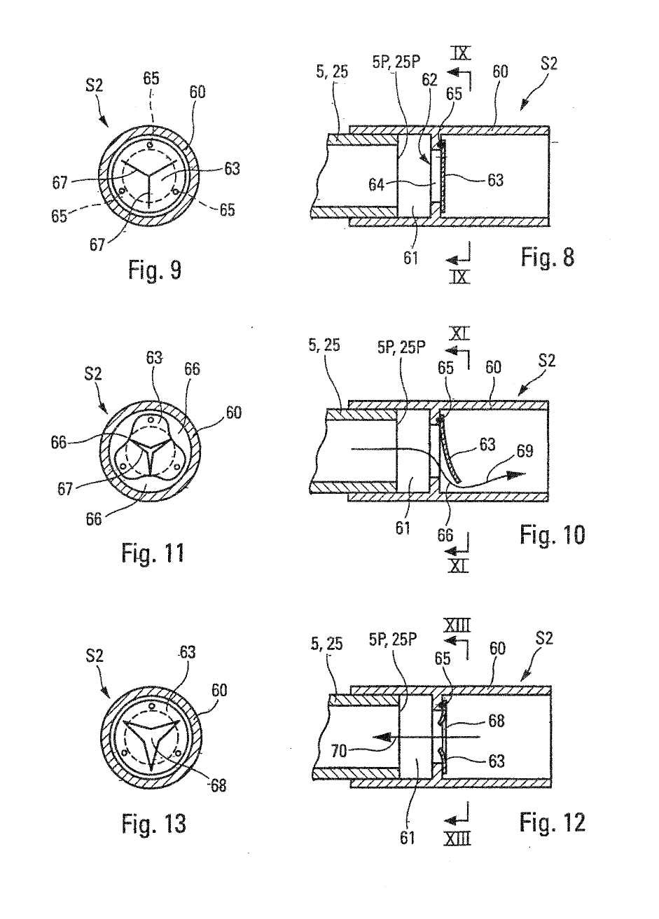

FIGS. 8, 10 and 12 illustrate, in schematic axial section, three states of a second example of a system according to the present invention, intended to improve the devices for artificial respiration from FIGS. 1 and 4.

FIGS. 9, 11 and 13 are schematic transverse sections along lines IX-IX, XI-XI and XIII-XIII, respectively, in FIGS. 8, 10 and 12.

The breathing mask 1, shown in FIG. 1, comprises a rigid shell of generally truncated cone shape 2 that can be applied to the face of a person 3 by way of a cuff 4 that borders its peripheral opening. At the opposite end, said mask 1 is provided with a device for artificial respiration D1, comprising a rigid tubular element 5 which is integral with said shell 2 or is fitted onto a tubular projection 6 of said shell 2. The tubular element 5 serves as a gas inlet and outlet connector piece for the mask 1, its proximal end 5P being exposed to the ambient air, while its distal end 5D is situated on the mask 1.

The tubular element 5 forms an internal main channel 7 and it comprises, in the middle part, deflection means 8 directed toward the axis L-L of said channel 7. The purpose of the deflection means 8 is to ensure that the jets of respiratory gas J injected through peripheral auxiliary channels 9 are deflected in the direction of said axis of the main channel 7, said auxiliary channels 9 being supplied from an inlet connector piece 10 (see arrow F symbolizing the supply of respiratory gas) by way of a peripheral annular chamber 11, and said jets of respiratory gas thus converging toward a point of convergence C on the axis L-L of said main channel 7 (see also FIGS. 2 and 3).

Moreover, the tubular element 5 comprises a connector piece 12 for removing gas and/or measuring pressure.

The variant embodiment D2 of the device for artificial respiration, shown in FIG. 4, comprises a flexible tubular element 25 that can form a nosepiece or mouthpiece, the distal end 25D of said tubular element 25 being designed to be introduced into an airway of a patient, while the proximal end 25P of the element 25 remains outside said patient. The tubular element 25 forms an internal main channel 27 and comprises deflection means 28 directed toward the axis L-L of said channel 27. The purpose of the deflection means 28 is to ensure that the jets of respiratory gas J injected through auxiliary channels 29 are deflected in the direction of said axis L-L, said auxiliary channels 29 being supplied from an inlet conduit 30 (see arrow F) by way of a peripheral chamber 31, said jets of respiratory gas J thereby converging toward a point of convergence C on said axis L-L.

Particularly when the devices for artificial respiration D1 and D2 are used for emergency resuscitation of persons who are in a state of cardiac arrest and whose thoracic cage is subjected to alternate compressions and decompressions, the devices are supplied continuously with respiratory gas (arrow F) from bottles of compressed gas or the like, and the jets J are continuous throughout the resuscitation process.

To avoid the disadvantages of residual positive pressure that were mentioned above, each of the devices D1 and D2 is equipped, at its proximal end 5P, 25P, with means for slowing down the intake of outside air into the tubular element 5, 25 at the beginning of each decompression. These means for slowing down the air can take different forms, in particular those designated by reference sign S1 (FIGS. 5 to 7) or reference sign S2 (FIGS. 8 to 13) and described hereinbelow. They can either form an integral part of the tubular element 5, 25 or can be attached releasably to the latter.

The means S1 for slowing down the intake of outside air into the tubular element 5, 25 at the beginning of each decompression, which means are shown in FIGS. 5 to 7, comprise a hollow body 40 delimiting a cavity 41 in free gaseous communication with the tubular element 5, 25. Fitted on the hollow body 40 are two valves 42 and 43, which are normally closed and are arranged in parallel between the cavity 41 and the outside. The valve 42 is designed to open spontaneously and immediately once the pressure inside the cavity 41 exceeds the external atmospheric pressure. By contrast, the valve 43 is designed to open spontaneously and gradually when the external atmospheric pressure exceeds the pressure inside the cavity 41. In the case shown in FIGS. 5 to 7, where said valves 42 and 43 are of the type with a head 44 or 45 applied against a seat 46 or 47 by the action of a spring 48 or 49, the immediate or gradual nature of the opening of said valves is achieved by control of the force of said springs 48, 49.

During a compression of the thoracic cage of the person 3 fitted with the device D1, D2, the pressure increases in the cavity 41, such that the valve 42 opens immediately, allowing the air expelled by the lungs of said person to escape freely, as is illustrated in FIG. 6 where the expelled air is symbolized by the arrows 50. During this compression, the valve 43 remains closed.

Conversely, during a decompression of the thoracic cage of said person 3, the pressure decreases in the cavity 41, such that the valve 43 opens gradually, allowing the air inspired from the outside to enter, while being slowed down, in the direction of the lungs of said person 3, as is symbolized by the arrows 51 in FIG. 7. During this decompression, the valve 42 remains closed.

Thus, the gradual and slowed down intake of outside air into the lungs of the person 3 allows the residual positive pressure due to the jets J to disappear at the beginning of the decompression.

The means S2 for slowing down the intake of outside air into the tubular element 5, 25 at the beginning of each decompression, which means are shown in FIGS. 8 to 13, comprise a hollow tubular body 60 delimiting a cavity 61 in free gaseous communication with the tubular element 5, 25. Fitted in the tubular body 60 is a valve 62, which is normally closed and is formed by an elastic membrane 63 bearing on a seat 64. The elastic membrane 63 is fixed to the seat 64 via several points of attachment 65 (of which 3 in the drawings) distributed about its periphery. The valve 62 is designed to open spontaneously and immediately once the pressure inside the cavity 61 exceeds the external atmospheric pressure, such opening of the valve 62 being due to the elastic membrane 63 coming away from the seat 64 between the points of attachment 65, on account of the deformation of said membrane under the effect of the pressure in the cavity 61. Passages 66 are thus created between the membrane 63 and the seat 64.

Moreover, slits 67 with contiguous edges are formed in the elastic membrane 63 and are able to form a valve that can open spontaneously, but gradually, when the external atmospheric pressure exceeds the pressure inside the cavity 61. In this case, a passage 68 forms spontaneously in the elastic membrane 63, by deformation of the latter, thus causing the contiguous edges of the slits 67 to move gradually apart.

During a compression of the thoracic cage of the person 3 fitted with the device D1, D2, the pressure increases in the cavity 61, such that the membrane 63 deforms with creation of the passages 66, thus allowing the air expelled by the lungs to escape freely to the outside (see arrow 69 in FIGS. 10 and 11).

During a decompression of the thoracic cage of the person 3, the pressure decreases in the cavity 61 and the elastic membrane 63 bears on its seat 64, thus closing the passages 66. By contrast, the contiguous edges of the slits 67 move gradually apart under deformation of the membrane 63, such that they form a passage 68 allowing the air inspired from the outside to enter, while being slowed down, in the direction of the lungs of said person, as is symbolized by the arrow 70 in FIG. 12.

1-8. (canceled)

9. A method for resuscitating a person in a state of cardiac arrest, according to which method alternate compressions and decompressions are exerted on the thoracic cage of said person, wherein:
a device is used that comprises:
a tubular element (5, 25) which forms a main channel (7, 27),
peripheral auxiliary channels (9, 29) which open into said main channel, said auxiliary channels being supplied continuously with respiratory gas, and
deflection means (8, 28) for causing the jets of respiratory gas (J) injected continuously through said auxiliary channels (9, 29) to converge on one another inside said main channel (7, 27),
in such a way that said main channel (7, 27) is connected via its distal end to an airway of said person (3), while the proximal end (5P, 25P) of said tubular element is located outside said person, such that the latter's respiratory system is connected to the outside by way of said main channel (7, 27), and
the intake of outside air into said tubular element (5, 25) is slowed down at the beginning of each decompression.

10. A device for carrying out the resuscitation method as claimed in claim 9, comprising:
a tubular element (5, 25) which forms a main channel (7, 27) and which is designed to be connected via its distal end to an airway of said person (3), while the proximal end (5P, 25P) of said tubular element is located outside said person and the latter's respiratory system is connected to the outside by way of said main channel (7, 27);
peripheral auxiliary channels (9, 29) which open into said main channel, said auxiliary channels being supplied continuously with respiratory gas; and
deflection means (8, 28) for causing the jets of respiratory gas (J) injected continuously through said auxiliary channels (9, 29) to converge on one another inside said main channel (7, 27),
wherein it comprises means (S1, S2) for spontaneously slowing down the intake of outside air into said tubular element (5, 25) at the beginning of each decompression.

11. The device as claimed in claim 10, wherein said means (S1, S2) for slowing down the intake of outside air into said tubular element (5, 25) comprise a hollow body (40, 60) provided with first and second valves (42, 43; 62, 67) which are normally closed, said first valve (42, 62) being able to open spontaneously and immediately during the compressions, while the second valve (43, 67) is able to open spontaneously, but gradually, during the decompressions, and in that said hollow body (40, 60) is arranged at the proximal end of said tubular element (5, 25).

12. The device as claimed in claim 11, wherein said first and second valves (42, 43) are arranged in parallel between the outside and the internal cavity (41) of said hollow body (40).

13. The device for artificial respiration as claimed in claim 11, wherein said first and second valves (62, 67) are arranged in series between the outside and the internal cavity (61) of said hollow body (60), one of said hollow valves being supported by the other.

14. The device for artificial respiration as claimed in claim 13, wherein said first valve (62) is formed by an elastic membrane (63) bearing spontaneously against a seat (64) provided in said hollow body (60) and being connected to said seat via several attachment points (65) distributed about its periphery, and the air expelled during the compressions passes freely from the cavity (61) of the hollow body (60) to the outside via passages (66) which form spontaneously and immediately through the elastic deformation of said membrane (63) between said points of attachment (65) and said seat (64), and in that said second valve is formed by at least one slit (67) with contiguous edges made in said membrane (63), and the air inspired during the decompressions passes gradually, being slowed down, from the outside to the cavity (61) of the hollow body (60) via the passage (68) which forms spontaneously in said membrane through elastic deformation thereof, causing its contiguous edges to move gradually apart.

15. The device for artificial respiration as claimed in claim 10, wherein said means (Si, S2) for slowing down the intake of outside air into said tubular element (5, 25) form an integral part of the latter.

16. The device for artificial respiration as claimed in claim 10, wherein said means (S1, S2) for slowing down the intake of outside air into said tubular element (5, 25) are attached releasably to the latter.
